(19) United States
(12) Patent Application Publication
(10) Pub. No.: US 2024/0252712 A1
(43) Pub. Date: Aug. 1, 2024

(54) PORTABLE BIOGENIC IONIZER WITH CONFIGURABLE DISPERSAL PATTERN

(71) Applicant: Best Technologies, Inc., Reno, NV (US)

(72) Inventor: John Chris Karamanos, San Jose, CA (US)

(21) Appl. No.: 18/595,671

(22) Filed: Mar. 5, 2024

Related U.S. Application Data
(63) Continuation-in-part of application No. 17/223,654, filed on Apr. 6, 2021.

(51) Int. Cl. A61L 9/22; B01D 53/32

(52) U.S. Cl. CPC A61L 9/22; B01D 53/32; B01D 2257/708; B01D 2257/91; B01D 2259/4508

(57) ABSTRACT

An improved biogenic ionizer with reduced acoustics and air entrainment is disclosed. A housing of the ionizer can comprise a group of apertures or vents through which ionized air is dispersed. Based on the configuration of the group of vents, ionized airflows can provided in a wide range of dispersal patterns that can vary between about 10 degrees to 360 degrees about the ionizer. The dispersal pattern can significantly improve the application of ions throughout a zone or space.

### CROSS-REFERENCE TO RELATED APPLICATIONS

This application is a continuation-in-part of, and claims priority to, U.S. application Ser. No. 17/223,654, filed Apr. 6, 2021, entitled “DEVICES AND SYSTEMS FOR CONCENTRATED BIOGENIC IONIZATION”. The entire contents of this disclosure is hereby incorporated by reference for all purposes, as if fully set forth herein.

### TECHNICAL FIELD

This disclosure relates generally to a portable biogenic ionizer that can discharge ionized air flows with positive and/or negative ions into a breathable space, and in some embodiments a portable unit that can be implemented into substantially any zone or space with the ionized discharge having an angle of coverage that can be configured according to the dimensions of the zone or space and/or to provide ionized discharge to more of the dimensions of a given zone or space.

### BACKGROUND

Devices for generating biogenic ionization for removing aerosolized pathogens, pollens, contaminants, and biologics in a closed space are known in the art. These devices generate an electric charge at a point or across a grid and draw air or pass air over the electrified point or grid. Positive and negative ions (cations and anions) are continuously created in the process and the ions travel in the airflow. This ionized airflow can neutralize pathogens or contaminants, provided the ions can come in contact with the pathogens or contaminants.

### BRIEF DESCRIPTION OF THE DRAWINGS

Numerous aspects, embodiments, objects, and advantages of the present embodiments will be apparent upon consideration of the following detailed description, taken in conjunction with the accompanying drawings, in which like reference characters refer to like parts throughout, and in which:

FIG. 1 is a front perspective view of one embodiment of an example portable and mountable ion dispensing system in accordance with certain embodiments of this disclosure;

FIG. 2 is a cut away partial view of an air flow passage with the air flow separator with electrodes or brushes in place in accordance with certain embodiments of this disclosure;

FIG. 3 is top-down orthogonal view of the air flow separator showing a cation brush or electrode on one side of the air flow separator and an anion electrode or brush on a second side of the air flow separator in accordance with certain embodiments of this disclosure;

FIG. 4 is a top-down isometric perspective view of the air flow separator of FIG. 3 with a bi-polar ionization unit and electrodes or brushes in place in accordance with certain embodiments of this disclosure;

FIG. 5 is a another top-down isometric perspective view of the air flow separator showing multiple different dispersal patterns as the ionized airflows exit vents in the housing as shown in FIG. 1 in accordance with certain embodiments of this disclosure;

FIG. 6 is a schematic top-down orthogonal view for a 360 degree dispersal pattern showing two connected V-shaped air flow separators that form a diamond shape and having multiple instances of cation and anion producing electrodes in accordance with certain embodiments of this disclosure;

FIG. 7 is a front view of an Air Handling Unit with numerous bi-polar ion generators at various locations along an air flow path;

FIG. 8 is a view of the plate to cover the Air Handling Unit;

FIG. 9 is a rear view of the Air Handling Unit of FIG. 7;

FIG. 10 is a rear view of the ion dispensing system of FIG. 1 with the Air Handling Unit of FIGS. 7-9 attached;

FIG. 11 is an example, non-limiting computing environment in which one or more embodiments described herein can be implemented;

FIG. 12 is an example, non-limiting networking environment in which one or more embodiments described herein can be implemented.

### DETAILED DESCRIPTION

### Overview

As noted in the Background section, ionized airflows can neutralize pathogens, volatile organic compounds (VOCs), or other contaminants provided the ions make contact with those pathogens, VOCs, or contaminants. Certain other systems attempt to implement a biogenic ionizer within an air handler unit (AHU) with the goal of turning the entire heating, ventilation, and air conditioning (HVAC) system into a biogenic ionizer solution as well. However, this approach may have certain drawbacks.

For example, positively charged cations are electrically attracted to negatively charged anions. Thus, if the cations and anions travel together, such as when traveling through HVAC ductwork, the ions will tend to intersect and cancel one another out, which can significantly reduce the efficacy of the process before the ions are able to reach a breathable space where intended to work.

In addition to ions interacting with each other, other ions may contact a wall or other surface without interacting with a contaminant particle, pathogen or other biologic. Ionizing the air in a typical AHU loses its overall effectiveness by only cleaning a small portion of the air in the breathe area.

Typically, ions only have a life expectancy of 5-60 seconds with a limited life cycle. Ions bond to metal, insulation and other surfaces, further reducing their effectiveness unless directly on a room surface, where some benefit may be realized. When installed in an AHU, the ions travel across the coils, through the duct work, metal turning vanes, the air terminal metal damper(s), and then through an air diffuser which may not have the proper isothermal uniform distribution pattern needed for full area coverage. Other factors such as stack effect when heating, stratification and stagnation zones in the area due to air distribution design factors affect system efficiency. Past attempts to generate and disperse ions have not been entirely satisfactory for enhancing indoor air quality (IAQ), which is set forth by the International Electrotechnical Commission (IEC), while reducing so-called ‘sick building syndrome’ (SBS). There is a continuing need for devices and systems that are portable (e.g., not implemented in an AHU), create a large number of anions and a rapid airflow into a breathable space without widespread deionization caused by the Coanda effect and other factors.

Prior Corona ionization systems use an electrical current to create bipolar ionized air. The Corona ionizer applies a high-voltage electrical current composed of a flow of negatively charged electrons, to a metal prong or needle. Electrostatic repulsion causes the electrons to detach from the prong or needle, attaching themselves to the molecules of nitrogen and oxygen in the air, forming negative ions, which are attracted to the static charge in the work environment thus neutralizing the ions. These ions also attract certain types of molecules in the work environment like dust and other air particulates. These particulates cluster around the ion, weighing it down and forcing it to fall to the ground thereby cleaning the air.

Corona ionization can further be divided into AC and DC types. AC or alternating current ionization uses one emitter to produce both positive and negative ions. This type of ionization is mainly used to protect components during assembly. DC direct current uses separate positive and negative power supplies that run simultaneously to create bipolar ions. DC ionizers are more efficient at producing ions and use lower operating currents, making them a better fit for cleanroom applications. The electrodes detailed herein can rely on any suitable type of ionization principle.

Cation and anion generators can include an oscillation signal generating circuit, a boost transformer, or a high voltage rectifying circuit. An input end of the oscillation signal can be connected to the power source and the primary electrode(s) of the boost transformer, which in turn can be connected to the oscillation signal output end of the oscillation generating circuit. The input end of the high voltage rectifying circuit can be connected with a secondary electrode of the boost voltage. An output end of the high voltage rectifying circuit can be respectively connected with a negative high voltage discharge and a positive high voltage discharge electrode. Cation and anion generators permit simultaneous generation of anions and cations.

The disclosed subject matter is now described with reference to the drawings, wherein like reference numerals are used to refer to like elements throughout. In the following description, for purposes of explanation, numerous specific details are set forth in order to provide a thorough understanding of the disclosed subject matter. It may be evident, however, that the disclosed subject matter may be practiced without these specific details. In other instances, well-known structures and devices are shown in block diagram form in order to facilitate describing the disclosed subject matter.

All figures and examples herein are intended to be non-limiting; they are mere exemplary iterations and/or illustrative embodiments of the claims appended to the end of this description. Modifications to specifically described devices, systems, the order of steps in processes, etc., are contemplated. The dispensing devices, systems and methods are capable of other embodiments and of being practiced and carried out in various ways. Also, it is to be understood that the phraseology and terminology employed herein are for the purpose of the description and should not be regarded as limiting. Moreover, discussion of the related art throughout the specification should in no way be considered as an admission that such related art is widely known or forms part of common general knowledge in the field.

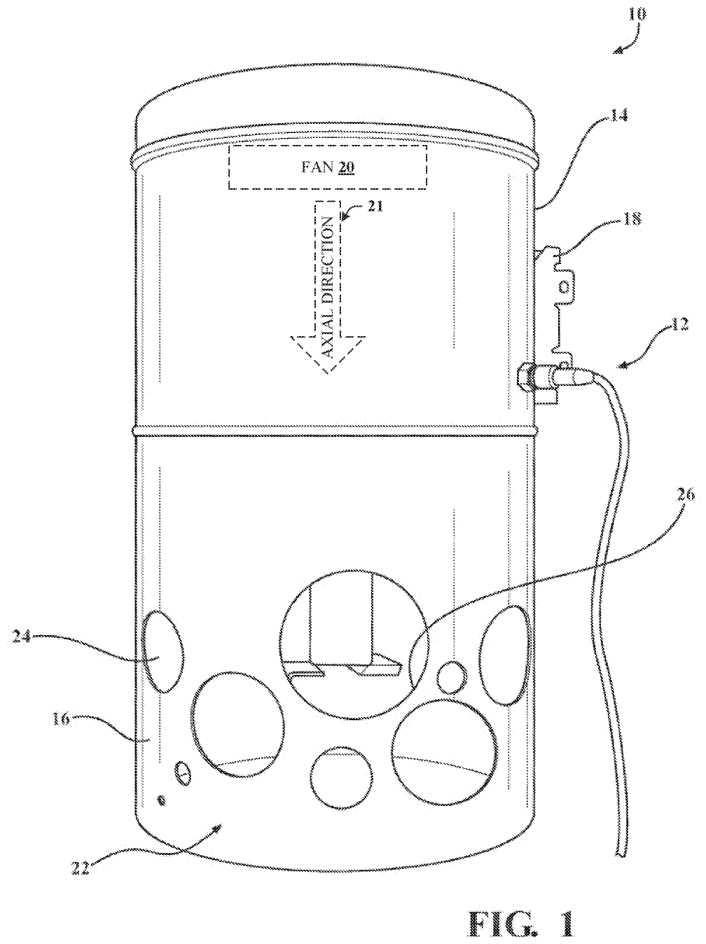

Turning now to the drawings wherein like numbers refer to like structures, FIG. 1 is a perspective front view of one embodiment of a portable ion dispensing device 10 having a housing or body 12, comprising first end 14 (e.g., a top portion) and opposing second end 16 (e.g., a bottom portion). The body 12 has one or more mounts 18, whereby the portable ion dispensing device may be mounted in place, potentially in any suitable location within a zone or space that is to be supplied with ionized flows. As shown, the body 12 is cylindrical, but it is understood any shaped body may be provided. The device 10 may be portable, or device 10 may be affixed in place as the user may desire.

The second end 16 of the body 12 can include a vent portion 22, having variously shaped vents 24 to facilitate desired circulation of ionized air. The vents 24 are shown as circular vents 26, but may be of any suitable shape having any suitable dimensions. Depending upon the airflow desired, it is contemplated the vents 24 may be of any configuration as long as adequate air flow is maintained through the device 10.

At first end 14, device 10 can comprise a fan 20. In other words, in the present embodiment, fan 20 can be situated in an upper or top portion of the housing or body 12, and can be configured to provide an airflow substantially in axial direction 21, that flows toward the lower or bottom portion (e.g., second end 16). Fan 20 can be a variable or multi-speed fan, a mixed flow fan, or any other suitable type of fan. The various speed or other settings of fan 20 can be configured to control any or all of the output of ions, the range and/or coverage distance of the ions, or the acoustics or noise-level of device 10. For example, fan 20 can have one or more nominal modes that operate with very little noise and have moderate ion dispersion and distance as well as one or more purge modes that have increased range and ion dispersion, but may create more noise. As an illustrative example, a purge mode can be selected for enhanced ion dispersal when an affected zone or space is unoccupied. In some embodiments, device 10 can receive a signal that indicates whether a particular space is unoccupied. The signal can be received from one or more sensors or other suitable devices, which can be implemented in the device 10 or received from a remote source.

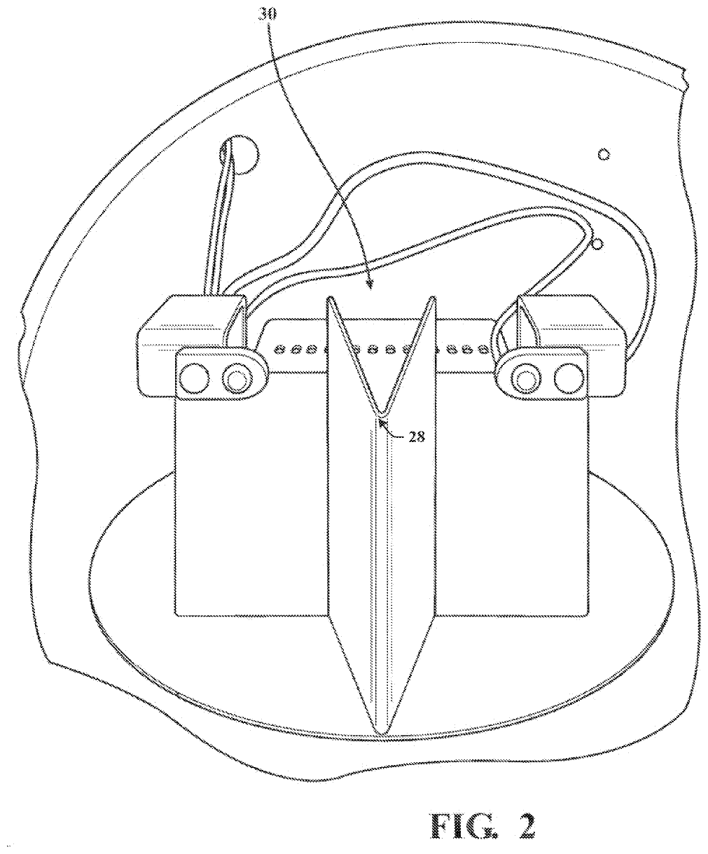

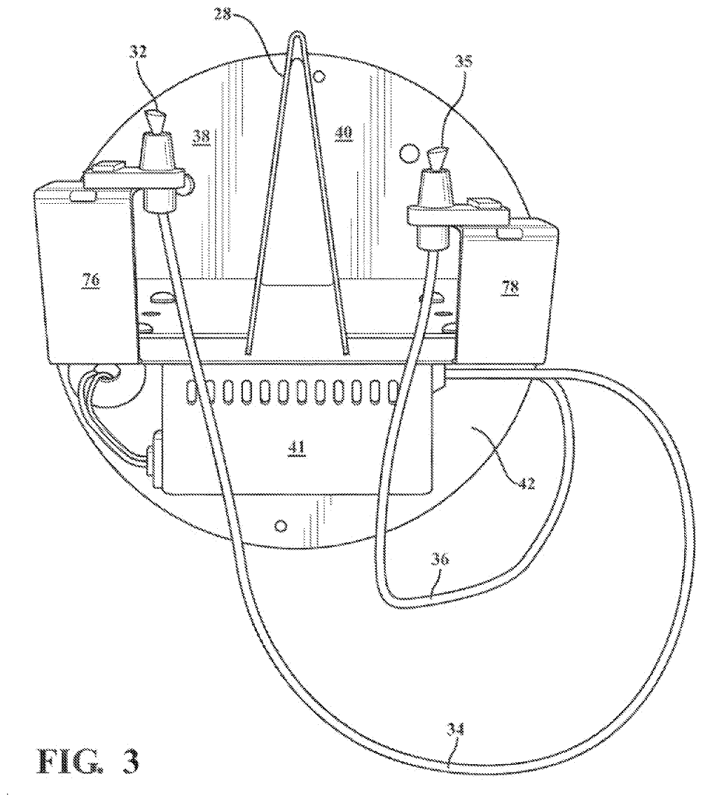

Turning to FIGS. 2-5, there is shown a V-shaped air flow separator 28 in the air passage 30. Air passage 30 can be provided by a housing or body 12 and can constrain the flow from fan 20 that flows substantially in the axial direction 21 toward V-shaped air flow separator 28. As shown in FIG. 3 (e.g., a top-down or orthogonal view), V-shaped air flow separator 28 can be configured to separate the flow of air within air passage 30 into separate streams or flow. On a first side 38 of the air flow separator 28, a first brush or electrode 32 is disposed or located and on the second side 40 of the air flow separator 28 a second brush or electrode 35 is disposed or located. Each set of the brushes or electrodes 32, 35 is separated from the other set by the air flow separator 28, which represents a divider that extends at least partially along the air passage 30. The brushes or electrodes 32, 35 can be electrically connected via wires 34, 36 or another electrically coupling technique to a cation and to anion bi-polar generator 41.

Anions can be generated on the first side 38 (e.g., by electrode(s) 32) and cations can be generated at the second side 40 (e.g., by electrode(s) 35). In some embodiments, the bi-polar generator 41 can be affixed to device 10. The bi-polar generator 41 may be any of the many on the market such as, for example, those available from Plasma Air of Hartford Conn. Those skilled in the art understand that the anions and cations may just as easily be generated on the opposite sides of the air flow separator 28 than as shown, depending upon the cation anion generator wiring to the electrodes 32, 35. In some embodiments, a given electrode 32, 35 can generate cations according to a first setting and anions according to a second setting, such as by a switching procedure at bi-polar generate 41. Hence, a given electrode 32, 35 can be implemented to produce cations for a first period, then switch to anion production for a second period, which can operate to disperse different polarity ions, in sequence, to a given portion of a zone or space.

A stop plate 42 may be provided so the air flow separator 28 may be mounted at the second end 16 (e.g., lower portions) of the body 12 of device 10. Stop plate 42 can also operate to direct air flowing through device 10 to ultimately be dispersed via vents 24. For example, stop plate 42 can largely stop the flow air through device 10 in the axial direction 21 and direct the air out through vents 24.

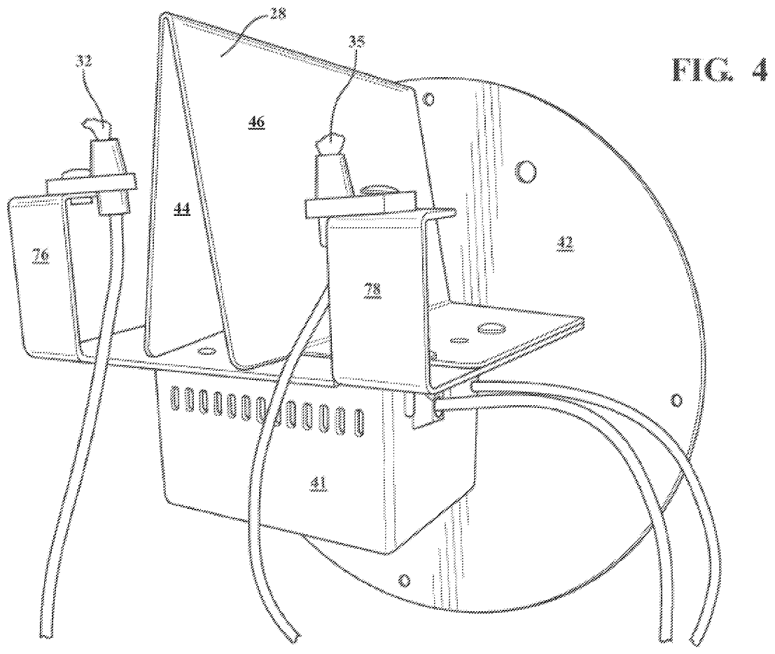

As shown at FIGS. 3 and 4, disposed in and along the first side 38 can be a first set of electrodes or brushes 32 which may be include one or more electrodes or brushes. A second set of electrodes or brushes 35, which may be one or more electrodes or brushes, is disposed in and along said second side 40 of the air flow separator 28. As shown, the electrodes are located at first and second flanges 76 and 78, respectively. The cationic and anionic generator can generate cations substantially along one side of the air flow separator 28 and can generate anions substantially on the other side air flow separator 28. As air is forced through the device 10, the cations and anions move in separate air flow passages and streams so that the life of the ions can be greatly extended as the ions do not immediately interact with each other while being generated within device 10. The air flow separator 28 can allow for maximizing ion output by separating the negative and positive electrodes so the ions respectively generated do not bind, thereby maximizing the total ion count that is dispersed from the device 10. The air flow separator 28 can facilitate changes in the air entrainment pattern as the V shaped separator throws out more ionized air per application.

The air flow separator with brushes may be positioned near the air flow openings (e.g., vents 24) of the device 10 to further maximize ion output. Such an arrangement can place the brushes and/or electrodes at the maximum pressure point of the fan to generate the most efficient ion output.

The air flow separator 28 can be a roughly “V” shaped article with sidewalls 44 and 46 oriented at acute angles relative to each other. While a V shape is shown having a particular acute angle, it is understood the separator could have walls oriented in a range of from about 5 degrees to 180 degrees relative to each other.

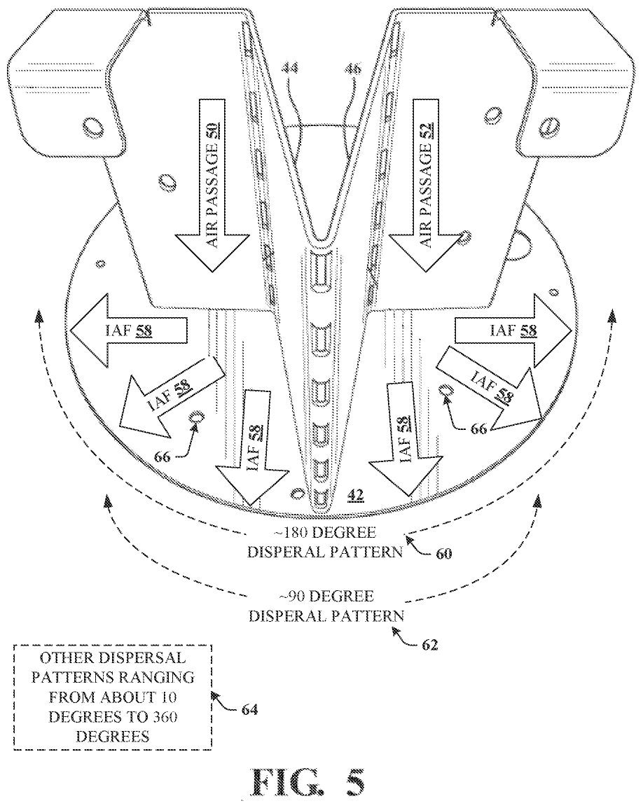

As illustrated at FIG. 5, sidewall 44 is shown intersecting sidewall 46, forming a vertex of the V shape in the vicinity of housing or body 12. Such operates to form two separate air passages, namely air passage 50 and air passage 52, which are in fluid communication with the ambient air being moved through and/or by fan 20. As air passages 50, 52 are downstream of electrodes 32, 35, air passages 50, 52 propagate ionized flows, one of which can contain cations and the other of which can contain anions. In this manner, each ionized flow can be physically separated from one another so that the different polarity ions do not attract and cancel out. Moreover, ions that contact sidewalls 44, 46, will each only contact a particular side 44, 46 that is shared with ions of the same polarity. Thus, should charge build on one of the sidewalls 44, 46, such will operate to repel addition ions from contacting the sidewalls 44, 46. In some embodiments, sidewalls 44, 46 can be electrically insulated from one another by any suitable means or technique.

Sidewalls 44, 46 can be coupled to or in contact with stop plate 42 such that ionized air flows 58 will be dispersed through vents 24 that are arranged along the lower portion of housing or body 12. The dispersal pattern can thus be subject to a number of, dimensions of, and locations of vents 24. In this example, vents 24 can be distributed across a 180 degrees arc of body 12 (e.g., as shown at FIG. 1), and therefore the coverage area of ionized air flows 58 can be dispersed into the zone or space at angles up to or exceeding about 180 degrees around the location of device 10. Such can operate to provide ionized flows throughout the entirety of a zone or space.

It is further appreciated that while air passages 50, 52 generally flow in axial direction 21, ionized air flows 58 proceed in a radial direction due to the locations of vents 24 and the presence of stop plate 42. Thus, after the flow from fan 20 is separated into multiple flows (e.g., by air flow separator 28) and is ionized (e.g., by electrodes 32, 35), the ionized flow may turn from what is approximately the axial direction 21 to the radial direction, which can be substantially perpendicular to axial direction 21. Ionized airflow turning by approximately 75 to 100 degrees (e.g., substantially perpendicular) can provide acoustic advantages as well as ion dispersion advantages. Moreover, the wide dispersal pattern that can cover up to or exceeding 180 degrees around device 10 can provide significantly more effective coverage of an entire zone, room, or other space in which device 10 is being operated.

As one example, device 10 can be mounted (e.g., via mounts 18) on a wall. The wide dispersal pattern 60 (e.g., 180 degrees) can thus disperse ionized flows 58 to potentially every part of the zone or space in which device 10 is mounted. As another example, device 10 can be mounted in a corner of a zone, room, or other space. In this embodiment, vents 24 can be configured such that ionized airflows 58 cover approximately a 90 degree dispersal pattern 62 that can potentially disperse ions to all portions of a zone or space from a corner location.

Other configurations can exist as well, as illustrated by range of dispersal patterns 64, which can be between about 10 degrees to a full 360 degrees. Some common configurations can be approximately 10 degrees, approximately 30 degrees, approximately 45 degrees, approximately 90 degrees, approximately 150 degrees, approximately 180 degrees, approximately 270 degrees, approximately 330 degrees, or approximately 360 degrees. As can be appreciated, dispersal patterns on the lower end of the range (e.g., about 10 degrees to about 20 degrees or so) can be well-suited for corridors or hallways, whereas dispersal patterns greater than 180 degrees can be well-suited for ceiling mounted or pole mounted placement or other centrally located placement (e.g., on a desk or other structure). For instance, the operation of mounting device 10 in the center of a room or space can be readily accomplished due to the portable and mountable nature of certain embodiments of device 10.

In order to effectuate the wide range of different dispersal patterns (e.g., any pattern from about 5 or 10 degrees to a full 360 degrees), vents 24 can be distributed accordingly during manufacture in some embodiments. In other embodiments, vents 24 can be configurable such as being movable by adjusting walls, screens, or other elements of the housing or body 12 to a desired arrangement, or providing a capability to open or close vents 24. In some embodiments, a screen element of body 12 can slide along the interior or exterior shape of the housing (in this example a cylinder), which can serve to cover or expose certain ones of vents 24 depending on location. In another embodiment, vents 24 can have adjustable screens such that vents 24 can be covered or exposed individually.

In some embodiments, stop plate 42 can be configured with holes 66, as illustrated. While vents 24 are typically configured to disperse ionized flows 58 in a radial direction (e.g., horizontally), small amounts of ionized flows can be dispersed substantially in axial direction 21 (e.g., vertically) via holes 66. In this manner, portions of the zone or space directly below device 10 can be supplied with ions. Such can provide additional coverage for areas that might not otherwise be covered by the radial dispersion of ionized flows 58, which can be particularly useful for wall, corner, or ceiling mounted implementations. In some embodiments, device 10 can be mounted in a rotated position such that the axial direction 21 is horizontal. Such can be useful for hallways or corridors or other narrow zones or spaces.

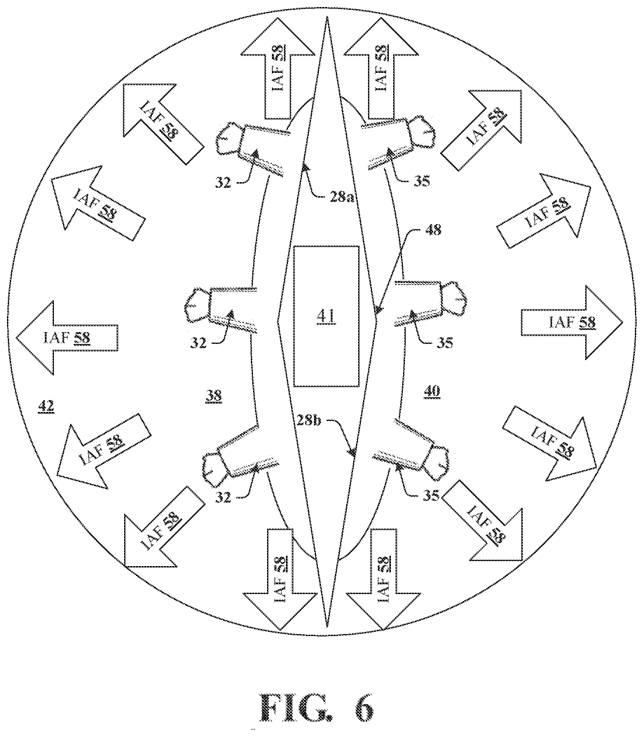

FIG. 6 is a schematic top-down orthogonal view for a 360 degree dispersal pattern showing two connected V-shaped air flow separators 28 that form a diamond-shaped spectator 48 and having multiple instances of cation and anion producing electrodes in accordance with certain embodiments of this disclosure.

As shown, in this embodiment, an upper V-shaped air flow separator 28a is coupled to or situated adjacent to a lower V-shaped air flow separator 28b. Together these two V-shaped air flow separators 28 form diamond-shape, the structure of which is referred to herein as air flow separator 48. While depicted here as a diamond shape (e.g., a rhombus or parallelogram), it is appreciated that any suitable shape is contemplated. Any shape that serves to separate air passage 30, which in this illustration flows into the page, into two or more separate streams can be suitable. For example, in some embodiments, air flow separator 48 can be two approximately parallel walls, but it can be seen that the diamond shape substantially maximizes the available locations of vents 24, thereby maximizing the potential coverage for the available dispersal patterns.

As illustrated, bi-polar generator 41 can be situated in a region inside air flow separator 48 and can control the production of ions by electrodes 32, 35 as described. Hence, in this embodiment, first side 38 and second side each respectively form a semi-circle, encompassing about 180 degrees of the interior of device 10. First side 38 can have multiple electrodes 32 that produce cations, while second side 40 can have multiple electrodes 35 that produce anions. These ions can be dispersed via ionized air flows 58 in a complete 360 degree dispersal pattern. Furthermore, in some embodiments, stop plate 42 can comprise holes 66 to provide ion dispersal in an axial direction 21 (e.g., into the page) as well.

In another embodiment, the unit may include a control module with integrated or remote sensor hub that senses Volatile Organic Compounds (VOCs) and/or CO2 levels, temperature, humidity, speakers, motion, occupancy, sound, or lights such as LED configured and run by a user's phone application or through a base building system. The sensor may also be configured to modulate fan speed based upon sensed occupancy of the breathe space or in disinfect mode based upon the size of the breathe space or area. The device may further be configured to operate on voice recognition command or gesture recognition. The sensors may also operate to permit the device to optimize ion output commensurate with the total breathe space diffusion in an isometric pattern as well as based on environmental factors such as an occupancy analysis or a use-case classification (e.g., the function of a given zone or space).

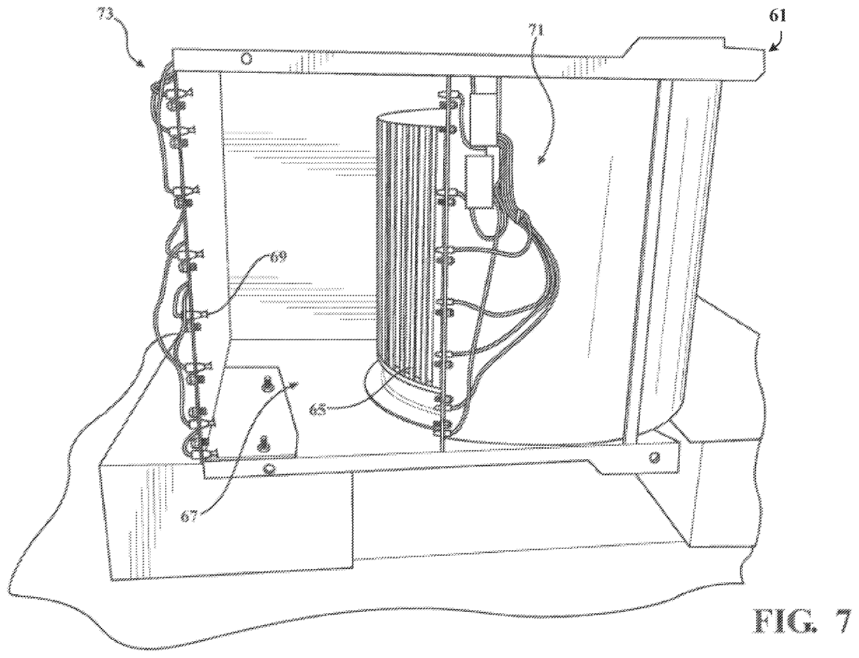

FIG. 7 shows a central AHU with various bipolar generators with brushes located in spaced apart relation to each other in the air passageway. Specifically, AHU 61 has a body with a squirrel cage blower type fan 65 housed within. Air flow passage 67 has various brushes 69 in spaced apart relation to each other along opposed first and second sides 71, 73, respectively, of the AHU. Each brush is electrically connected to a bi-polar generator or biogenic ionizer, and the generators/ionizers electrically connectable to a power source. Cations are created at one side of the air passageway, and anions are created at the opposite side of the passageway. When the squirrel cage fan is activated, ionized air flows through the AHU.

The electrodes or brushes, regardless of invention embodiment described, may be cleaned by oscillating the brushes. In this regard, it is contemplated the electrodes may be extended into or sheathed in a material, (e.g., metal or plastic or other suitable material) such as if in a drinking straw, so that as air blows across the brushes that oscillate to increase cleaning action and removal of debris. This may be accomplished by turning off the ion generator and increasing the fan speed to maximum CFM to cause the brushes to oscillate and self-clean. In another embodiment, it is contemplated to leave the ion generator operational and oscillate the brushes with the force of air blowing across them. In the latter case, the higher the air speed across the brushes, the more the brushes may oscillate.

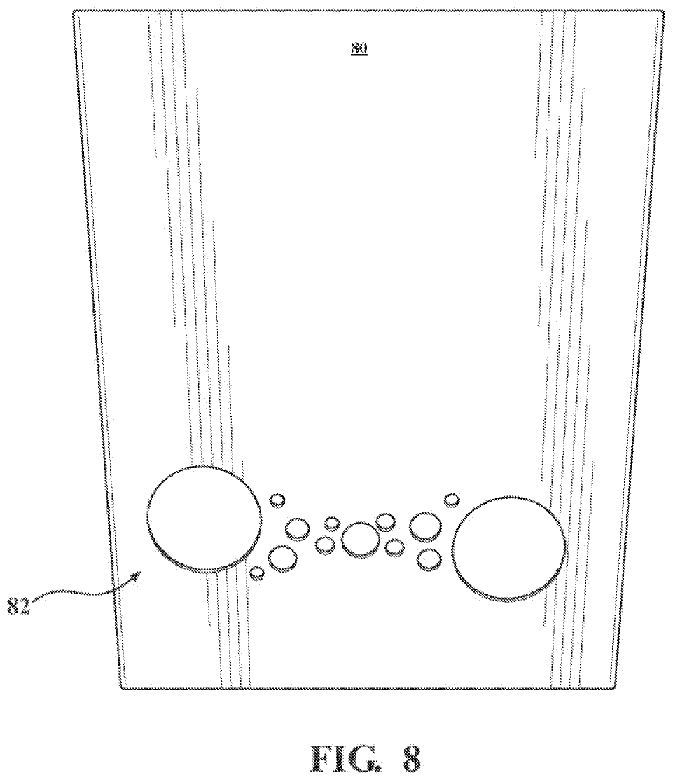

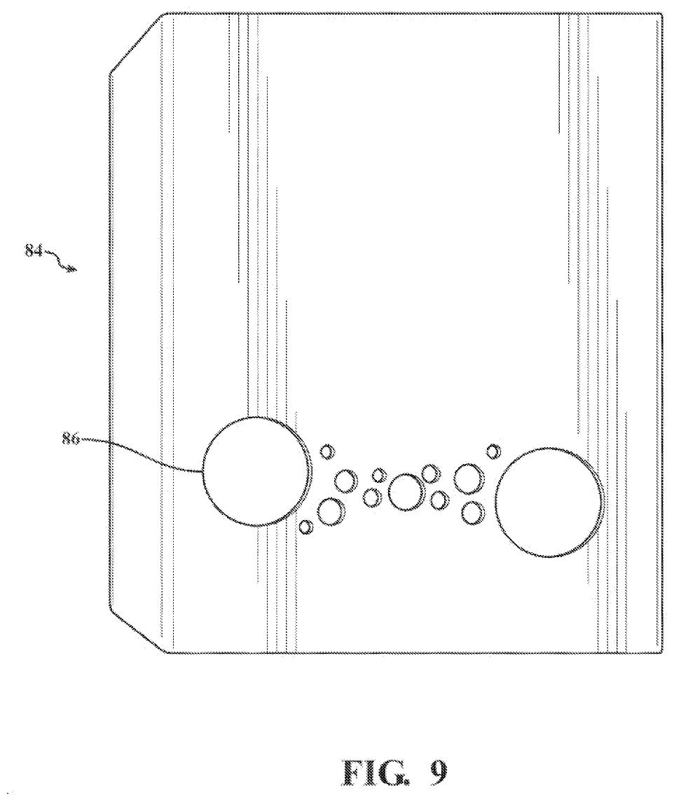

FIGS. 8-10 show one embodiment of an AHU that includes the biogenic ionizer of the present application. Plate 80 is a cover equipped with air outlet apertures 82. The plate, which may be removable, can be a wall of the controller box 84. The box may include the biogenic ionizer as well as the air passages and air flow separators. The controller box can be attachable to the body 12 of the device 10, and can include air outlet apertures 86 (e.g., similar to vents 24) on one or more sides or salient surfaces. The outlet apertures 86 may be positioned or situated in fluid communication with apertures in the body 12 and air is drawn through the air outlet apertures 82 upon activation of the unit. Specifically, the biogenic ionizer is activated, and creates cation and anions within separate air flow passages as previously described. A fan in the body draws the ionized air through the unit and disburses it through vents 24.

The device as described may further be equipped with damper devices to vary the air flow/velocity though the openings 82. The damper may be a plate within the body such that rotation of the plate inside the unit progressively covers the outlet holes, thereby increasing velocity of air flow through the smaller holes. Rotating the plate in an opposite direct will increase the size of the opening, thereby increasing air flow through and decreasing air velocity.

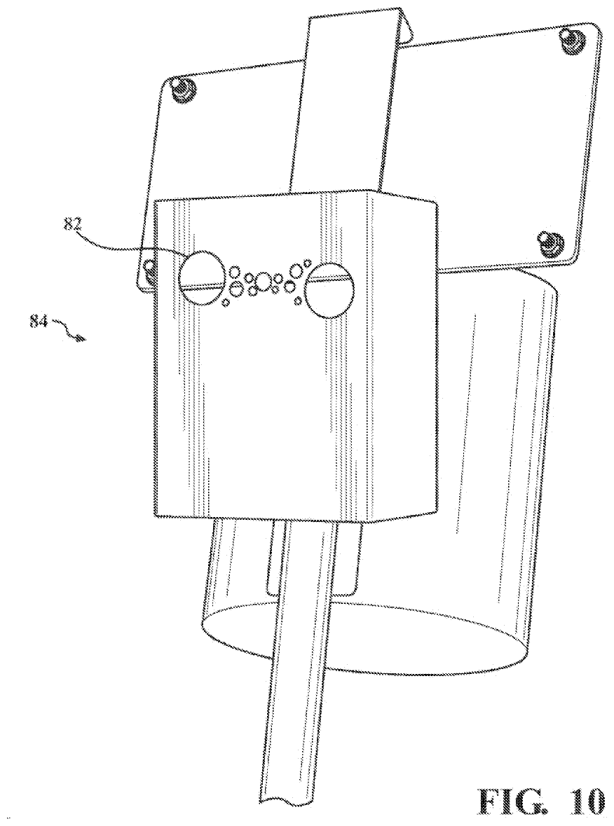

FIG. 10 shows the apparatus may be mounted on a pole for positioning at any height in a closed space such as a room.

The device and systems as described may be used in a wide variety of applications and may be operated singly, in serial or in parallel with each other, depending upon the requirements of treating a breathe space or multiple breath spaces. The devices can be modulated such that it can be configured to a variety of breathe spaces. Different breath spaces require differing air flow to disinfect a given space. The actual cubic feet per minute (CFM) airflow may be determined for a given space and the operation of the device modulated to create that CFM. It is anticipated the device may be used in areas where as little as 125 CFM is required to as much as 2000 CFM is required to disinfect a given breathe space.

### EXAMPLES

### Example 1

A 600 square foot room was saturated with glycol vapor to simulate smoke contamination. An air meter was calibrated to ambient air conditions. The biogenic ionizer was located in close proximity to the ceiling of the room. The ionizer was activated and saturated the air in the 600 square foot room with 300,000 ion cc/minute at breathing zones for 17 minutes. The biogenic ionizer had a variable fan and moved the air through the biogenic ionizer to exit at about 1500 cfm. During the 17 minute period after start of ionization, the following was observed:

Glycol vapor was clearing by 5 minutes after start and was visibly cleared by 8 minutes after start of ionization.

Room air quality was restored to ambient outside air quality within 17 minutes of start of ionization. An Ion meter in the room showed the ion count progressively dropped for 8 minutes after start of ionization as the glycol vapor was removed from the air. During the next 9 minutes after start of ionization, the ion count in the room air progressively increased, indicating the glycol vapor had been progressively ionized and removed from the room air. The ion count plateaued at 17 minutes after start of ionization at a level consistent with ion generation levels from the biogenic ionizer indicating the glycol vapor was substantially removed from the room air.

With regard to the processes described herein, it should be understood that, although the steps of such processes, have been described as occurring in a certain sequence, such processes could be practiced with the described steps performed in an order other than the exemplary order. It further should be understood that certain steps could be performed simultaneously, that other steps could be added, or that certain steps described herein could be omit-ted. In other words, the descriptions of processes herein are provided for the purpose of illustrating certain embodiments and should in no way be construed so as to limit the claimed invention.

Accordingly, it is to be understood that the above description is intended to be illustrative and not restrictive. Many embodiments and applications other than the examples provided would be apparent upon reading the above description. The scope of the invention should be determined with reference to the appended claims along with the full scope of equivalents to which such claims are entitled. It is anticipated and intended that future developments will occur, and that the disclosed systems and processes will be incorporated into such future embodiments. The invention is capable of modification and variation.

All terms used in the claims are intended to be given their broadest reasonable constructions and their ordinary meanings as understood by those knowledgeable in the technologies described herein unless an explicit indication to the contrary is made herein.

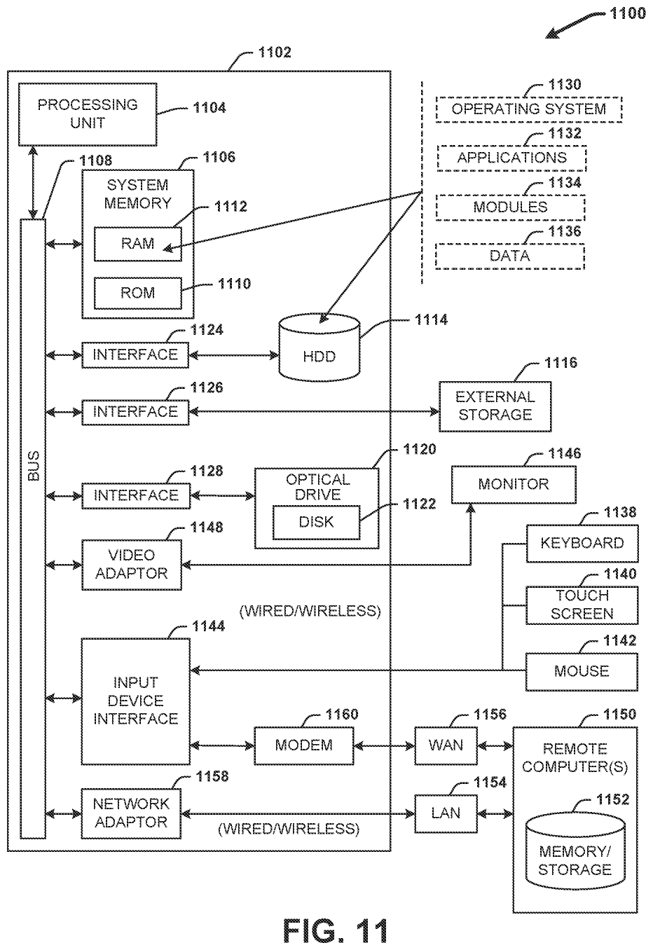

In order to provide additional context for various embodiments described herein, FIG. 11 and the following discussion are intended to provide a brief, general description of a suitable computing environment 1100 in which the various embodiments of the embodiment described herein can be implemented. While the embodiments have been described above in the general context of computer-executable instructions that can run on one or more computers, those skilled in the art will recognize that the embodiments can be also implemented in combination with other program modules and/or as a combination of hardware and software.

Generally, program modules include routines, programs, components, data structures, etc., that perform particular tasks or implement particular abstract data types. Moreover, those skilled in the art will appreciate that the various methods can be practiced with other computer system configurations, including single-processor or multiprocessor computer systems, minicomputers, mainframe computers, Internet of Things (IoT) devices, distributed computing systems, as well as personal computers, hand-held computing devices, microprocessor-based or programmable consumer electronics, and the like, each of which can be operatively coupled to one or more associated devices.

The illustrated embodiments of the embodiments herein can be also practiced in distributed computing environments where certain tasks are performed by remote processing devices that are linked through a communications network. In a distributed computing environment, program modules can be located in both local and remote memory storage devices.

Computing devices typically include a variety of media, which can include computer-readable storage media, machine-readable storage media, and/or communications media, which two terms are used herein differently from one another as follows. Computer-readable storage media or machine-readable storage media can be any available storage media that can be accessed by the computer and includes both volatile and nonvolatile media, removable and non-removable media. By way of example, and not limitation, computer-readable storage media or machine-readable storage media can be implemented in connection with any method or technology for storage of information such as computer-readable or machine-readable instructions, program modules, structured data or unstructured data.

Computer-readable storage media can include, but are not limited to, random access memory (RAM), read only memory (ROM), electrically erasable programmable read only memory (EEPROM), flash memory or other memory technology, compact disk read only memory (CD ROM), digital versatile disk (DVD), Blu-ray disc (BD) or other optical disk storage, magnetic cassettes, magnetic tape, magnetic disk storage or other magnetic storage devices, solid state drives or other solid state storage devices, or other tangible and/or non-transitory media which can be used to store desired information. In this regard, the terms “tangible” or “non-transitory”herein as applied to storage, memory or computer-readable media, are to be understood to exclude only propagating transitory signals per se as modifiers and do not relinquish rights to all standard storage, memory or computer-readable media that are not only propagating transitory signals per se.

Computer-readable storage media can be accessed by one or more local or remote computing devices, e.g., via access requests, queries or other data retrieval protocols, for a variety of operations with respect to the information stored by the medium.

Communications media typically embody computer-readable instructions, data structures, program modules or other structured or unstructured data in a data signal such as a modulated data signal, e.g., a carrier wave or other transport mechanism, and includes any information delivery or transport media. The term “modulated data signal” or signals refers to a signal that has one or more of its characteristics set or changed in such a manner as to encode information in one or more signals. By way of example, and not limitation, communication media include wired media, such as a wired network or direct-wired connection, and wireless media such as acoustic, RF, infrared and other wireless media.

With reference again to FIG. 11, the example environment 1100 for implementing various example embodiments described herein includes a computer 1102, the computer 1102 including a processing unit 1104, a system memory 1106 and a system bus 1108. The system bus 1108 couples system components including, but not limited to, the system memory 1106 to the processing unit 1104. The processing unit 1104 can be any of various commercially available processors. Dual microprocessors and other multi processor architectures can also be employed as the processing unit 1104.

The system bus 1108 can be any of several types of bus structure that can further interconnect to a memory bus (with or without a memory controller), a peripheral bus, and a local bus using any of a variety of commercially available bus architectures. The system memory 1106 includes ROM 1110 and RAM 1112. A basic input/output system (BIOS) can be stored in a non-volatile memory such as ROM, erasable programmable read only memory (EPROM), EEPROM, which BIOS contains the basic routines that help to transfer information between elements within the computer 1102, such as during startup. The RAM 1112 can also include a high-speed RAM such as static RAM for caching data.

The computer 1102 further includes an internal hard disk drive (HDD) 1114 (e.g., EIDE, SATA), one or more external storage devices 1116 (e.g., a magnetic floppy disk drive (FDD) 1116, a memory stick or flash drive reader, a memory card reader, etc.) and an optical disk drive 1120 (e.g., which can read or write from a CD-ROM disc, a DVD, a BD, etc.). While the internal HDD 1114 is illustrated as located within the computer 1102, the internal HDD 1114 can also be configured for external use in a suitable chassis (not shown). Additionally, while not shown in environment 1100, a solid state drive (SSD) could be used in addition to, or in place of, an HDD 1114. The HDD 1114, external storage device(s) 1116 and optical disk drive 1120 can be connected to the system bus 1108 by an HDD interface 1124, an external storage interface 1126 and an optical drive interface 1128, respectively. The interface 1124 for external drive implementations can include at least one or both of Universal Serial Bus (USB) and Institute of Electrical and Electronics Engineers (IEEE) 1194 interface technologies. Other external drive connection technologies are within contemplation of the embodiments described herein.

The drives and their associated computer-readable storage media provide nonvolatile storage of data, data structures, computer-executable instructions, and so forth. For the computer 1102, the drives and storage media accommodate the storage of any data in a suitable digital format. Although the description of computer-readable storage media above refers to respective types of storage devices, it should be appreciated by those skilled in the art that other types of storage media which are readable by a computer, whether presently existing or developed in the future, could also be used in the example operating environment, and further, that any such storage media can contain computer-executable instructions for performing the methods described herein.

A number of program modules can be stored in the drives and RAM 1112, including an operating system 1130, one or more application programs 1132, other program modules 1134 and program data 1136. All or portions of the operating system, applications, modules, and/or data can also be cached in the RAM 1112. The systems and methods described herein can be implemented utilizing various commercially available operating systems or combinations of operating systems.

Computer 1102 can optionally comprise emulation technologies. For example, a hypervisor (not shown) or other intermediary can emulate a hardware environment for operating system 1130, and the emulated hardware can optionally be different from the hardware illustrated in FIG. 11. In such an embodiment, operating system 1130 can comprise one virtual machine (VM) of multiple VMs hosted at computer 1102. Furthermore, operating system 1130 can provide runtime environments, such as the Java runtime environment or the .NET framework, for applications 1132. Runtime environments are consistent execution environments that allow applications 1132 to run on any operating system that includes the runtime environment. Similarly, operating system 1130 can support containers, and applications 1132 can be in the form of containers, which are lightweight, standalone, executable packages of software that include, e.g., code, runtime, system tools, system libraries and settings for an application.

Further, computer 1102 can be enabled with a security module, such as a trusted processing module (TPM). For instance, with a TPM, boot components hash next in time boot components, and wait for a match of results to secured values, before loading a next boot component. This process can take place at any layer in the code execution stack of computer 1102, e.g., applied at the application execution level or at the operating system (OS) kernel level, thereby enabling security at any level of code execution.

A user can enter commands and information into the computer 1102 through one or more wired/wireless input devices, e.g., a keyboard 1138, a touch screen 1140, and a pointing device, such as a mouse 1142. Other input devices (not shown) can include a microphone, an infrared (IR) remote control, a radio frequency (RF) remote control, or other remote control, a joystick, a virtual reality controller and/or virtual reality headset, a game pad, a stylus pen, an image input device, e.g., camera(s), a gesture sensor input device, a vision movement sensor input device, an emotion or facial detection device, a biometric input device, e.g., fingerprint or iris scanner, or the like. These and other input devices are often connected to the processing unit 1104 through an input device interface 1144 that can be coupled to the system bus 1108, but can be connected by other interfaces, such as a parallel port, an IEEE 1194 serial port, a game port, a USB port, an IR interface, a BLUETOOTH® interface, etc.

A monitor 1146 or other type of display device can be also connected to the system bus 1108 via an interface, such as a video adapter 1148. In addition to the monitor 1146, a computer typically includes other peripheral output devices (not shown), such as speakers, printers, etc.

The computer 1102 can operate in a networked environment using logical connections via wired and/or wireless communications to one or more remote computers, such as a remote computer(s) 1150. The remote computer(s) 1150 can be a workstation, a server computer, a router, a personal computer, portable computer, microprocessor-based entertainment appliance, a peer device or other common network node, and typically includes many or all of the elements described relative to the computer 1102, although, for purposes of brevity, only a memory/storage device 1152 is illustrated. The logical connections depicted include wired/wireless connectivity to a local area network (LAN) 1154 and/or larger networks, e.g., a wide area network (WAN) 1156. Such LAN and WAN networking environments are commonplace in offices and companies, and facilitate enterprise-wide computer networks, such as intranets, all of which can connect to a global communications network, e.g., the Internet.

When used in a LAN networking environment, the computer 1102 can be connected to the local network 1154 through a wired and/or wireless communication network interface or adapter 1158. The adapter 1158 can facilitate wired or wireless communication to the LAN 1154, which can also include a wireless access point (AP) disposed thereon for communicating with the adapter 1158 in a wireless mode.

When used in a WAN networking environment, the computer 1102 can include a modem 1160 or can be connected to a communications server on the WAN 1156 via other means for establishing communications over the WAN 1156, such as by way of the Internet. The modem 1160, which can be internal or external and a wired or wireless device, can be connected to the system bus 1108 via the input device interface 1144. In a networked environment, program modules depicted relative to the computer 1102 or portions thereof, can be stored in the remote memory/storage device 1152. It will be appreciated that the network connections shown are example and other means of establishing a communications link between the computers can be used.

When used in either a LAN or WAN networking environment, the computer 1102 can access cloud storage systems or other network-based storage systems in addition to, or in place of, external storage devices 1116 as described above. Generally, a connection between the computer 1102 and a cloud storage system can be established over a LAN 1154 or WAN 1156 e.g., by the adapter 1158 or modem 1160, respectively. Upon connecting the computer 1102 to an associated cloud storage system, the external storage interface 1126 can, with the aid of the adapter 1158 and/or modem 1160, manage storage provided by the cloud storage system as it would other types of external storage. For instance, the external storage interface 1126 can be configured to provide access to cloud storage sources as if those sources were physically connected to the computer 1102.

The computer 1102 can be operable to communicate with any wireless devices or entities operatively disposed in wireless communication, e.g., a printer, scanner, desktop and/or portable computer, portable data assistant, communications satellite, any piece of equipment or location associated with a wirelessly detectable tag (e.g., a kiosk, news stand, store shelf, etc.), and telephone. This can include Wireless Fidelity (Wi-Fi) and BLUETOOTH® wireless technologies. Thus, the communication can be a predefined structure as with a conventional network or simply an ad hoc communication between at least two devices.

Wi-Fi, or Wireless Fidelity, allows connection to the Internet from a couch at home, a bed in a hotel room, or a conference room at work, without wires. Wi-Fi is a wireless technology similar to that used in a cell phone that enables such devices, e.g., computers, to send and receive data indoors and out; anywhere within the range of a base station. Wi-Fi networks use radio technologies called IEEE 802.11 (a, b, g, n, etc.) to provide secure, reliable, fast wireless connectivity. A Wi-Fi network can be used to connect computers to each other, to the Internet, and to wired networks (which use IEEE802.3 or Ethernet). Wi-Fi networks operate in the unlicensed 5 GHz radio band at a 54 Mbps (802.11a) data rate, and/or a 2.4 GHz radio band at an 11 Mbps (802.11b), a 54 Mbps (802.11g) data rate, or up to a 600 Mbps (802.11n) data rate for example, or with products that contain both bands (dual band), so the networks can provide real-world performance similar to the basic “10BaseT” wired Ethernet networks used in many offices.

As it employed in the subject specification, the term “processor” can refer to substantially any computing processing unit or device comprising, but not limited to comprising, single-core processors; single-processors with software multithread execution capability; multi-core processors; multi-core processors with software multithread execution capability; multi-core processors with hardware multithread technology; parallel platforms; and parallel platforms with distributed shared memory in a single machine or multiple machines. Additionally, a processor can refer to an integrated circuit, a state machine, an application specific integrated circuit (ASIC), a digital signal processor (DSP), a programmable gate array (PGA) including a field programmable gate array (FPGA), a programmable logic controller (PLC), a complex programmable logic device (CPLD), a discrete gate or transistor logic, discrete hardware components, or any combination thereof designed to perform the functions described herein. Processors can exploit nano-scale architectures such as, but not limited to, molecular and quantum-dot based transistors, switches and gates, in order to optimize space usage or enhance performance of user equipment. A processor may also be implemented as a combination of computing processing units. One or more processors can be utilized in supporting a virtualized computing environment. The virtualized computing environment may support one or more virtual machines representing computers, servers, or other computing devices. In such virtualized virtual machines, components such as processors and storage devices may be virtualized or logically represented. In an example embodiment, when a processor executes instructions to perform “operations”, this could include the processor performing the operations directly and/or facilitating, directing, or cooperating with another device or component to perform the operations.

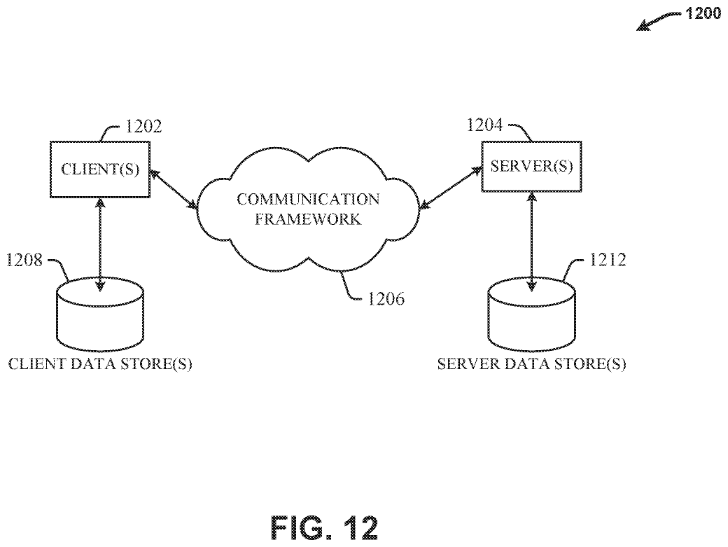

Referring now to FIG. 12, there is illustrated a schematic block diagram of a computing environment 1200 in accordance with this specification. The system 1200 includes one or more client(s) 1202, (e.g., computers, smart phones, tablets, cameras, PDA's). The client(s) 1202 can be hardware and/or software (e.g., threads, processes, computing devices). The client(s) 1202 can house cookie(s) and/or associated contextual information by employing the specification, for example.

The system 1200 also includes one or more server(s) 1204. The server(s) 1204 can also be hardware or hardware in combination with software (e.g., threads, processes, computing devices). The servers 1204 can house threads to perform transformations of media items by employing aspects of this disclosure, for example. One possible communication between a client 1202 and a server 1204 can be in the form of a data packet adapted to be transmitted between two or more computer processes wherein data packets may include coded analyzed headspaces and/or input. The data packet can include a cookie and/or associated contextual information, for example. The system 1200 includes a communication framework 1206 (e.g., a global communication network such as the Internet) that can be employed to facilitate communications between the client(s) 1202 and the server(s) 1204.

Communications can be facilitated via a wired (including optical fiber) and/or wireless technology. The client(s) 1202 are operatively connected to one or more client data store(s) 1208 that can be employed to store information local to the client(s) 1202 (e.g., cookie(s) and/or associated contextual information). Similarly, the server(s) 1204 are operatively connected to one or more server data store(s) 1210 that can be employed to store information local to the servers 1204. Further, the client(s) 1202 can be operatively connected to one or more server data store(s) 1210.

In one exemplary implementation, a client 1202 can transfer an encoded file, (e.g., encoded media item), to server 1204. Server 1204 can store the file, decode the file, or transmit the file to another client 1202. It is noted that a client 1202 can also transfer uncompressed file to a server 1204 and server 1204 can compress the file and/or transform the file in accordance with this disclosure. Likewise, server 1204 can encode information and transmit the information via communication framework 1206 to one or more clients 1202.

The illustrated aspects of the disclosure can also be practiced in distributed computing environments where certain tasks are performed by remote processing devices that are linked through a communications network. In a distributed computing environment, program modules can be located in both local and remote memory storage devices.

The above description includes non-limiting examples of the various embodiments. It is, of course, not possible to describe every conceivable combination of components or methods for purposes of describing the disclosed subject matter, and one skilled in the art can recognize that further combinations and permutations of the various embodiments are possible. The disclosed subject matter is intended to embrace all such alterations, modifications, and variations that fall within the spirit and scope of the appended claims.

With regard to the various functions performed by the above-described components, devices, circuits, systems, etc., the terms (including a reference to a “means”) used to describe such components are intended to also include, unless otherwise indicated, any structure(s) which performs the specified function of the described component (e.g., a functional equivalent), even if not structurally equivalent to the disclosed structure. In addition, while a particular feature of the disclosed subject matter may have been disclosed with respect to only one of several implementations, such feature can be combined with one or more other features of the other implementations as may be desired and advantageous for any given or particular application.

The terms “exemplary” and/or “demonstrative” as used herein are intended to mean serving as an example, instance, or illustration. For the avoidance of doubt, the subject matter disclosed herein is not limited by such examples. In addition, any aspect or design described herein as “exemplary” and/or “demonstrative” is not necessarily to be construed as preferred or advantageous over other aspects or designs, nor is it meant to preclude equivalent structures and techniques known to one skilled in the art. Furthermore, to the extent that the terms “includes,” “has,” “contains,” and other similar words are used in either the detailed description or the claims, such terms are intended to be inclusive-in a manner similar to the term “comprising” as an open transition word-without precluding any additional or other elements.

The term “or” as used herein is intended to mean an inclusive “or” rather than an exclusive “or.” For example, the phrase “A or B” is intended to include instances of A, B, and both A and B. Additionally, the articles “a” and “an” as used in this application and the appended claims should generally be construed to mean “one or more” unless either otherwise specified or clear from the context to be directed to a singular form.

The term “set” as employed herein excludes the empty set, i.e., the set with no elements therein. Thus, a “set” in the subject disclosure includes one or more elements or entities. Likewise, the term “group” as utilized herein refers to a collection of one or more entities.

The description of illustrated embodiments of the subject disclosure as provided herein, including what is described in the Abstract, is not intended to be exhaustive or to limit the disclosed embodiments to the precise forms disclosed. While specific embodiments and examples are described herein for illustrative purposes, various modifications are possible that are considered within the scope of such embodiments and examples, as one skilled in the art can recognize. In this regard, while the subject matter has been described herein in connection with various embodiments and corresponding drawings, where applicable, it is to be understood that other similar embodiments can be used or modifications and additions can be made to the described embodiments for performing the same, similar, alternative, or substitute function of the disclosed subject matter without deviating there from. Therefore, the disclosed subject matter should not be limited to any single embodiment described herein, but rather should be construed in breadth and scope in accordance with the appended claims below.

What is claimed is:

1. A system, comprising:
a housing configured to provide an air passageway for a flow of air received from a fan device in an axial direction;
an air flow separator, situated in the air passageway, configured to separate the flow into a first flow and a second flow, wherein the first flow traverses a first side of the air flow separator and the second flow traverses a second side of the air flow separator;
a first electrode, situated at the first side of the air flow separator, that generates cations, causing the first flow to become a first ionized flow comprising positively charged ions;
a second electrode, situated at the second side of the air flow separator, that generates anions, causing the second flow to become a second ionized flow comprising negatively charged ions; and
a group of apertures formed in the housing, the group of apertures comprising a first aperture configured to disperse a portion of the first ionized flow in a first radial direction and a second aperture configured to disperse a portion of the second ionized flow in a second radial direction, wherein an angle between the first radial direction and the second radial direction is at least 10 degrees.

2. The system of claim 1, further comprising a stop plate, situated downstream of the air flow separator, configured to cause respective portions of the first ionized flow and respective portions of the second ionized flow to traverse respective apertures of the group of apertures in respective radial directions that differ from the axial direction by at least 75 degrees.

3. The system of claim 2, wherein the stop plate comprises at least one vent configured to allow some portion of the first ionized flow or the second ionized flow to be exhausted substantially in the axial direction.

4. The system of claim 1, wherein the first flow traverses the first side substantially in the axial direction, and the second flow traverses the second side substantially in the axial direction.

5. The system of claim 1, wherein the group of apertures are configured according to a corner unit implementation and the angle between the first radial direction and the second radial direction is at least 75 degrees.

6. The system of claim 1, wherein the group of apertures are configured according to a wall unit implementation and the angle between the first radial direction and the second radial direction is at least 150 degrees.

7. The system of claim 1, wherein the group of apertures are configured according to a hanging unit implementation and the angle between the first radial direction and the second radial direction is at least 330 degrees.

8. The system of claim 1, wherein a cross section of the air flow separator has a V-shape when the cross section is taken at any intersection of a plane that is substantially perpendicular to the axial direction.

9. The system of claim 8, wherein an angle of the V-shape is configurable based on implementation.

10. The system of claim 8, wherein the angle of the V-shape ranges between five and 180 degrees.

11. The system of claim 1, further comprising the fan device.

12. The system of claim 1, wherein the fan device is a multi-speed fan or a mixed flow fan.

13. The system of claim 1, wherein the fan device comprises a purge mode setting that increases a speed of the fan above nominal operation.

14. The system of claim 13, wherein the purge mode setting is activated in response to a determination that a zone supplied by the first ionized flow or the second ionized flow is unoccupied.

15. The system of claim 1, further comprising multiple first electrodes situated at multiple first locations within the first flow.

16. A biogenic ionizer device, comprising:
a housing configured to provide an air passageway for a flow of air received from a fan device;
a group of electrodes, situated within the air passageway downstream of the fan device, that generates ions, causing the flow to become an ionized flow;
multiple apertures formed in the housing downstream of the group of electrodes, the multiple apertures configured to discharge the ionized flow into a breathable space in multiple different radial directions, wherein at least two of the multiple different radial directions differ by an angle of at least 45 degrees.

17. The biogenic ionizer device of claim 16, wherein the angle is substantially 180 degrees.

18. The biogenic ionizer device of claim 16, further comprising a stop plate, situated downstream of the group of electrodes, that causes the ionized flow that is flowing in an axial direction to turn at least 75 degrees before being discharged into the breathable space in the multiple different radial directions.

19. A biogenic ionizer device, comprising:
a housing configured to provide an air passageway for a flow of air received from a fan device in axial direction;
a group of electrodes, situated within the air passageway downstream of the fan device, that generates ions, causing the flow to become an ionized flow;
multiple apertures formed in the housing downstream of the group of electrodes, the multiple apertures configured to discharge, in multiple radial directions that are substantially perpendicular to the axial direction, the ionized flow into a breathable space.

20. The biogenic ionizer device of claim 19, wherein the multiple apertures span the housing radially over, causing the ionized flow to be discharged into the breathable space over an angle of at least one of: approximately 10 degrees, approximately 45 degrees, approximately 90 degrees, approximately 180 degrees, approximately 360 degrees, or any angle between approximately 10 degrees and 360 degrees.
